(19) United States
(12) Patent Application Publication
(10) Pub. No.: US 2005/0169990 A1
(43) Pub. Date: Aug. 4, 2005

(54) Controlled release dosage forms using acrylic polymer, and process for making

(76) Inventors: Huai-Hung Kao, Syosset, NY (US); Yadi Zeng, Fort Lee, NJ (US); Fai Jim, Franklin Square, NY (US); Sou-Chan Chang, Westbury, NY (US)

Correspondence Address: GUY DONATIELLO;ENDO PHARMACEUTICALS, 100 PAINTERS DRIVE, CHADDS FORD, PA, 19317, US

(21) Appl. No.: 10/501,798

(22) Filed: Jul. 19, 2004

Related U.S. Application Data
(63) Continuation of application No. PCT/US02/18088, filed on Jun. 7, 2002.
(60) Provisional application No. 60/297,150, filed on Jun. 8, 2001.

(51) Int. Cl. A61K 31/485; A61K 9/22

(52) U.S. Cl. 424/468; 514/282; 264/109

(57) ABSTRACT

Process for dry mixing a controlled release oral dosage form are provided. The dosage form is produced by mixing, tableting, and curing dosage forms. The cured dosage forms exhibit controlled release properties superior to those of uncured tablets.

### FIELD OF THE INVENTION

The present invention relates to controlled release dosage forms containing an acrylic polymer and a process for making the same.

### BACKGROUND OF THE INVENTION

Controlled release dosage forms of therapeutically active substances have advantages over conventional administration forms. These advantages include delaying drug absorption until it reaches a certain portion of the alimentary tract, where absorption of the drug is most therapeutically effective, and allowing the drug to be released slowly in the gastrointestinal tract, which prolongs the systemic action of the drug.

One major drawback of conventional administration of drug therapy is that it needs to be carefully monitored in order to maintain an effective steady state blood level of the drug. Otherwise, undesirable peaks and valleys in the plasma drug concentration can occur, which may interfere with the therapeutic activity of the treatment. An advantage of controlled release dosage forms is their ability to maintain optimal steady drug plasma levels with reductions in the frequency of administration. A further advantage of these dosage forms is the improvement of patient compliance, which is usually achieved by incurring fewer missed doses due to patient forgetfulness. Another advantage of controlled release dosage forms is the ability to tailor the release of a drug to a specific portion of the gastrointestinal tract. This will not only ensure that a certain concentration of the drug is released at the appropriate site, but also limits the amount of unnecessary drug exposure to unaffected areas.

One such method of obtaining controlled release dosage forms is by incorporating the drug into a polymer matrix. Polymers such as certain cellulose derivatives, zein, acrylic resins, waxes, higher aliphatic alcohols, and polylactic and polyglycolic acids have been used. In addition to mixing the drug with the polymer matrix, coating the drug with an appropriate polymer matrix has also been known to produce controlled release dosage forms, such as specially formulated coated beads or pellets, coated tablets, capsules, and coated ion-exchange resins. Different types of polymers/matrices are known in the pharmaceutical industry for controlling the release of active pharmaceutical ingredient from dosage forms, and the mechanism of each control is based on the characteristics of the polymer. In oral delivery matrices, the drug, when immersed in solution, diffuses through the polymer matrix and is released. In other matrices, the water-soluble ingredients dissolve when the dosage form is contacted with a dissolution medium, leaving behind a backbone of the undissolved matrix. Drugs in such situations release by migrating through the pores left behind by the dissolved ingredients.

In another dosage form, polymers may need to be treated before forming matrices with controlling mechanisms. This treatment usually involves heating the polymers, possibly above certain characteristic temperatures.

Two main conventional methods are known in the art for the preparation of materials to be included in a solid dosage form: wet processes and dry processes. Wet processes require the addition of water or organic solvent to the blend, forming a wet blend, prior to forming the dosage form. After being uniformly mixed, the formed granulate is then dried, in an oven, by fluid bed drying, or by any other conventional drying methods. Once the solvent has evaporated, the granules are milled or crushed in a manner so that particles of uniform particle size are formed. After milling or crushing, the granules are ready to be processed into a finish dosage form. One frequent problem encountered with wet granulation processes is the inability to detect or determine the end point of drying, without the granules being too dry or too wet for subsequent steps. In order to achieve the optimal drying process, tedious steps are built into manufacturing processes so that at various intervals during the drying stage, representative samples are taken and measured for the moisture content until an optimal amount is reached. This drying process is difficult to control, as the drying rate varies from run to run. In addition, the wet granulation processes are not suitable for all formulations. Active pharmaceutical ingredients may be moisture sensitive; the exposure to the solvents used in wet granulation processes may increase the degradation of the compounds. In summary, wet granulation processes are complicated, tedious and time-consuming.

Dry processes consist of dry granulation and direct compression. Dry granulation may be used where one of the constituents, either the drug or the diluent, has sufficient cohesive properties to form the finished dosage form. This process includes mixing the ingredients, slugging, dry screening, lubricating, and finally compressing the ingredients. In direct compression, the powdered materials to be included in the solid dosage form are compressed directly without modifying the physical nature of the material itself. It may consist of a series of dry blendings, whereby various ingredients are mixed with the active ingredient in a blender. The resulting blend may be passed through a roller compacter before milling, after which the blend is ready to be put into its fished dosage form. Because no solvent is introduced during the dry processes, these processes are particularly useful with moisture sensitive substances.

### SUMMARY OF TEE INVENTION

The present invention provides controlled release formulations and processes for obtaining controlled release dosage forms. “Dry” when used to describe embodiments of the present invention means that no solvent, water or organic solvents, are needed during the processes leading to obtaining a matrix for the dosage form. The dry methods involve dry mixing the active pharmaceutical ingredient(s) with an acrylic polymer and then forming and curing the dosage form. Forming can be done with drug granulation prior to compression or direct compression. Curing the dosage form produces an oral dosage form with a desirable, uniform, predictable, controlled release rate in an efficient and cost effective manner. The method can be used with a wide range of active pharmaceutical compounds and acrylic matrices. The preferred acrylic polymer is ammonio methacrylate copolymer.

### BRIEF DESCRIPTION OF THE DRAWINGS

FIG. 1 shows the dissolution profile of uncured and cored tablets of Example 1.

FIG. 2 shows the dissolution profile of uncured and cured tablets of Example 2.

FIG. 3 shows the dissolution profile of uncured and cured tablets of Example 3.

FIG. 4 shows the dissolution profile of uncured and cured tablets of Example 4.

FIG. 5 shows the dissolution profile of uncured and cured tablets of Example 5.

FIG. 6 is a Differential Scanning Calorimetry (DSC) thermogram of ammonio methacrylate copolymer (Eudragit®).

FIG. 7 is a DSC thermogram of the uncured tablet of Formulation 1 of Example 1.

FIG. 8 is a DSC thermogram of the cured tablet of Formulation 1 of Example 1.

FIG. 9 is a DSC thermogram of the uncured tablet of Formulation 2 of Example 2.

FIG. 10 is a DSC thermogram of the cured tablet of Formulation 2 of Example 2.

In the present invention, it was surprisingly found that directly dry mixing a blend containing an acrylic polymer and an active ingredient, without the addition of water or solvent, coupled with a curing process, provides dosage forms having controlled release properties.

A mixture is obtained by directly mixing the acrylic polymer with a therapeutically effective amount of an active ingredient A preferred acrylic polymer is ammonio methacrylate copolymer. Ammonio methacrylate copolymers of this type preferred for use herein are water-insoluble, swellable, film-forming polymers based on neutral methacrylic acid esters with a small proportion of trimethyl-ammonioethyl methacrylate chloride. Most particularly preferred is a polymer having a molar ratio of the quaternary ammonium groups to the neural ester groups of about 1:40 (corresponding to roughly 25 meq./100 g). One such polymer is sold under the name Eudragit® from Rohm America, Inc. of Piscataway, N.J. The polymer/active ingredient mixture preferably further includes excipients. Any generally acceptable pharmaceutical excipients can be used. Examples of such excipients are flavoring agents, lubricants, solubilizers, suspending agents, fillers, compression aids, binders, and encapsulating material. Specific suitable solid carries include calcium phosphate, magnesium stearate, talc, sugars, lactose, dextran, starch, gelatin, cellulose, methyl cellulose, sodium carboxymethyl cellulose, polyvinyl pyrrolidine, low melting waxes, and ion exchange carriers. Such carrier may be added before or after the tablet is compressed, as is well known in the art.

In a preferred embodiment, the acrylic polymer comprises from about 10% to about 90% of the dry weight of the mixture. More preferably, the acrylic polymer comprises from about 20% to about 80% of the dry weight of the mixture, more preferably from about 30% to about 70% of the dry weight of the mixture, and most preferably from about 30% to about 55% of the dry weight of the mixture.

The active ingredient may be any therapeutically active pharmaceutical ingredient(s) or a combination of active ingredients. Preferred active ingredients include opioids, including, but not limited to morphine, hydromorphone, codeine, oxycodone, oxymorphone, nalbuphine, hydrocodone, dihydrocodeine, dihydromorphine, buprenorphine, naltrexone, naloxone, salts of any of the foregoing, mixtures of any of the foregoing, and the like.

The mixture containing an active ingredient, an acrylic polymer, and any optional excipients is formed into a solid unit dosage form. Such processes include the preparation of the mixture and compression of the mixture into tablets. The resulting tablets are solid dosage forms of substantially homogenous composition. A lubricant may also be used. The tablet is a substantially uniform matrix, that may dissolve in a relatively uniform manner.

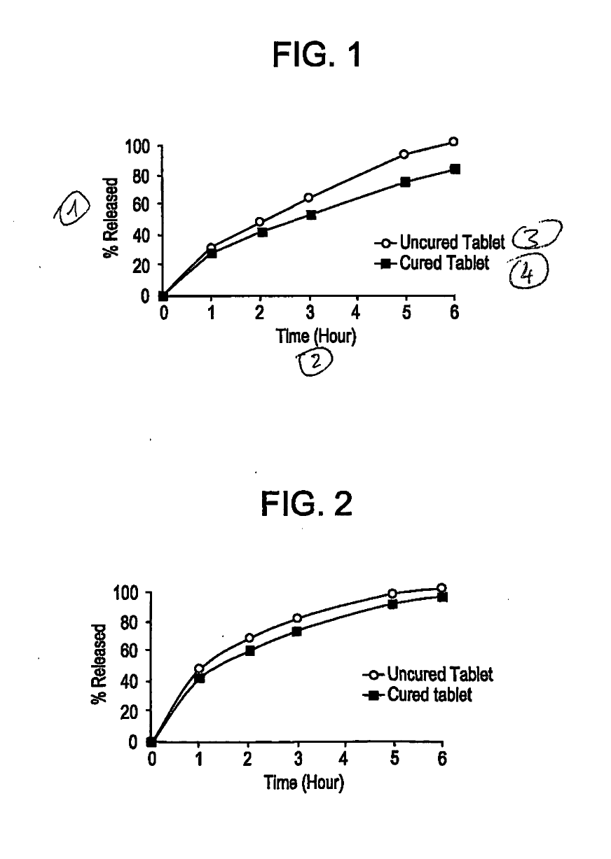

Such processes also include a curing step during manufacturing of the tablet. In a prefaced sequence of the process, the mixture is compressed, and the compressed mixture or tablet is then cured. Cured tablets of the present invention have been found to produce better control of the release of the active ingredients, as evidenced by more desirable dissolution profiles. As shown in FIG. 1, the release profile of the dosage form of the cured tablet was slower and more consistent than that of the uncured tablet.

To obtain cured tablets, the tablets are exposed to a temperature exceeding the curing temperature of the polymer. The temperature for which the tablet must be cured varies with the nature of the acrylic polymer used, as well as the composition and size of the dosage form. In the case of the preferred acrylic material set forth herein, temperatures in the rage of from about 40° C. to about 70° C. are appropriate. Preferably, a temperature of at least about 50° C. is used, more preferably at least about 55° C. Higher temperatures may be used, so long as the tablet (or more preferably at least about 55° C. Higher temperatures may be used, so long as the tablet (or active ingredient) remains unharmed. The time of curing varies with the temperature. Higher temperatures allow the tablet to cure faster. It is important that the entire tablet reach the cure temperature. The time required will therefore depend on the temperature of the oven (or coating pan, etc.), the desired core temperature for the polymer, and the tablet size, among other factors. Generally, the desired curing occurs between about 10 minutes and about one hour. Longer cure times are generally not harmful, unless the temperature is so high that damage to one or more components of the tablet occurs.

Although the tablets produced using the above process provide excellent controlled release characteristics, it may be desirable to further control the release of the active pharmaceutical ingredient through the use of a coating layer. Such a layer could be used to delay the initial release of the active pharmaceutical ingredient, for instance, until the tablet moves out of the stomach. Coating of dosage forms to obtain delayed release may be used in conjunction with the curing process described herein, and can be applied before or after the tablet is cured. Inks, dyes, and imprinting may also be applied to such tablets.

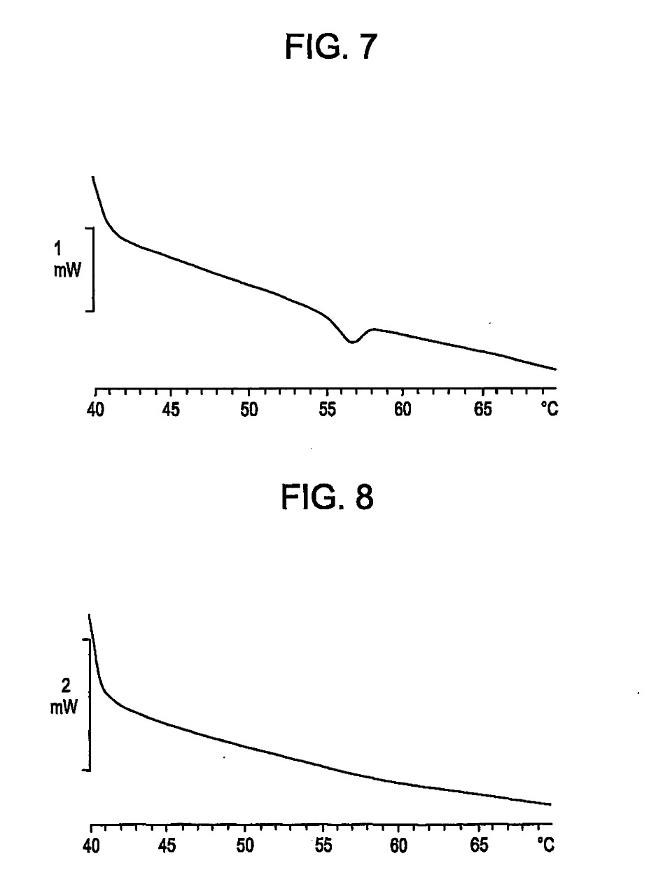

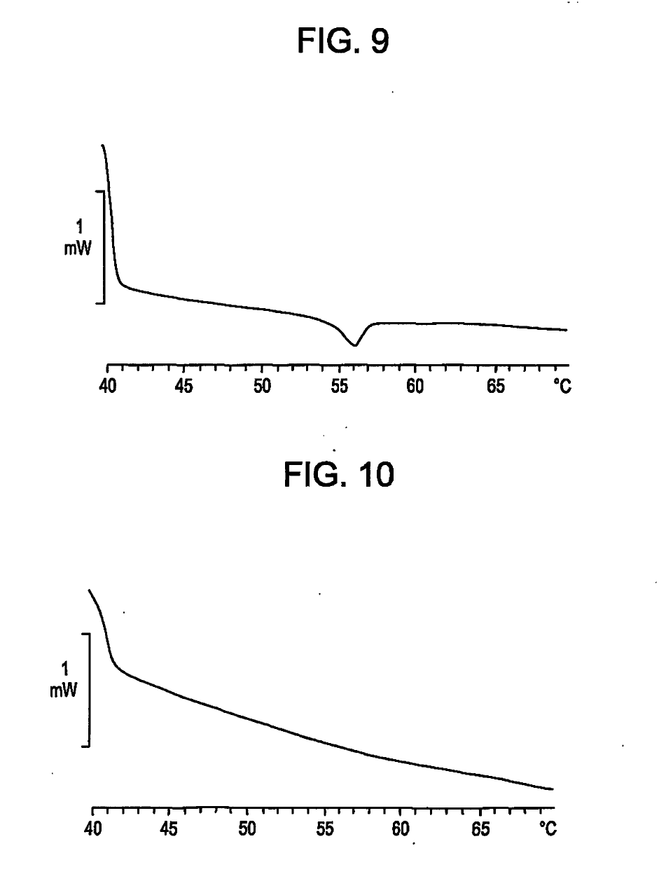

DSC results can be used to examine the difference in the release profiles of cured and uncured tablets. FIGS. 7 and 8 show DSC scans of uncured and cured tablets of Formulation 1. FIG. 7, taken before curing has a peak around 56° C. In contrast, the absence of the peak in this temperature area shown in FIG. 8 indicates that the tablets had been cured. Likewise, the uncured tablet of Formulation 2 shows a peak at 56° C. (FIG. 9) while the cured tablet has no peak in the same region (FIG. 10). As shown in FIGS. 1 and 2 and Tables 1A and 2A, cured tablets were able to release the drug in a more controlled manner producing slower and more consistent dissolution profiles.

The following examples illustrate various aspects of the present invention. They are not to be construed to limit the claims in any manner whatsoever.

### EXAMPLES

Oxycodone controlled release tablets were prepared by dry mixing the ingredients and directly compressing the blend into tablets. These tablets were then cured.

### Example 1

TABLE 1
Formulation 1

| | Tablet |
|---|---|
| Description | Composition (mg) |
| Oxycodone Hydrochloride | 40.000 |
| Microcrystalline Cellulose | 111.650 |
| Ammonio Methacrylate Copolymer | 225.000 |
| Colloidal Silicon Dioxide | 9.000 |
| Sodium Lauryl Sulfate | 18.000 |
| Magnesium Hydroxide | 1.350 |
| Povidone | 33.750 |
| Stearic Acid | 5.625 |
| Magnesium Stearate | 5.625 |
| Total Core Tablet Weight | 450.000 |
| Opadry Cosmetic Coating | 13.500 |
| Total Coated Tablet Weight | 463.500 |

Comparison of Cured and Uncured Tablets

Dissolution profiles for cured and uncured Formulation 1 tablets were obtained using the USP Basket Method (Type I Dissolution) at 100 rpm in 0.1N HCl at 37□° C. As seen from FIG. 1, uncured tablets were found to have rapid release profiles. When these same tablets were cured, it was surprisingly found that the release profiles become slower than before they were subjected to the elevated temperature. Table 1A below shows a comparison between the dissolution profiles of cured and uncured Formulation 1 tablets.

TABLE 1A
Dissolution Profiles of Uncured and Cured Formulation 1 Tablets:

| | Uncured Tablets | Cured Tablets % Active |
|---|---|---|
| Time (hr) | % Active Ingredient Released | Ingredient Released |
| 0 | 0.0 | 0.0 |
| 1 | 29.8 | 26.6 |
| 2 | 44.4 | 39.1 |
| 3 | 60.4 | 50.4 |
| 5 | 87.7 | 71.3 |
| 6 | 94.9 | 79.4 |
| 8 | 98.5 | 90.3 |
| 10 | 99.5 | 96.5 |
| 12 | 100.0 | 100.0 |

### Example 2

TABLE 2
Formulation 2

| | Tablet |
|---|---|
| Description | Composition (mg) |
| Oxycodone Hydrochloride | 40.000 |
| Microcrystalline Cellulose | 15.605 |
| Ammonio Methacrylate Copolymer | 82.500 |
| Colloidal Silicon Dioxide | 3.300 |
| Sodium Lauryl Sulfate | 6.600 |
| Magnesium Hydroxide | 0.495 |
| Povidone | 12.375 |
| Stearic Acid | 2.063 |
| Magnesium Stearate | 2.063 |
| Total Tablet Weight | 165.000 |
| Opadry Cosmetic Coating | 4.950 |
| Total Coated Tablet Weight | 169.950 |

TABLE 2A
Dissolution Profiles of Uncured and Cured Formulation 2 Tablets:

| | Uncured Tablets | Cured Tablets |
|---|---|---|
| | % Active Ingredient | % Active Ingredient |
| Time (hr) | Released | Released |
| 0 | 0.0 | 0.0 |
| 1 | 47.7 | 42.0 |
| 2 | 66.3 | 58.6 |
| 3 | 79.7 | 71.4 |
| 5 | 94.5 | 88.4 |
| 6 | 97.6 | 93.2 |
| 8 | 99.4 | 97.5 |
| 10 | 100.2 | 99.2 |
| 12 | 100.0 | 100.0 |

The dissolution data shown in Table 2A and illustrated in FIG. 2 showed that slower release profiles were obtained with cured tablets as opposed to uncured ones.

### Example 3

TABLE 3
Formulation 3

| | Tablet |
|---|---|
| Description | Composition (mg) |
| Oxycodone Hydrochloride | 10.000 |
| Microcrystalline Cellulose | 50.480 |
| Ammonio Methacrylate Copolymer | 56.700 |
| Colloidal Silicon Dioxide | 2.800 |
| Sodium Lauryl Sulfate | 5.600 |
| Magnesium Hydroxide | 0.420 |
| Povidone | 10.500 |
| Stearic Acid | 1.750 |
| Magnesium Stearate | 1.750 |
| Total Tablet Weight | 140.000 |
| Opadry Cosmetic Coating | 4.200 |
| Total Coated Tablet Weight | 144.200 |

TABLE 3A
Dissolution Profiles of Uncured and Cured Formulation 3 Tablets:

| | Uncured Tablets | Cured Tablets |
|---|---|---|
| | % Active Ingredient | % Active Ingredient |
| Time (hr) | Released | Released |
| 0 | 0.0 | 0.0 |
| 1 | 39.8 | 30.9 |
| 2 | 68.0 | 43.8 |
| 3 | 89.3 | 56.1 |
| 5 | 98.3 | 78.1 |
| 6 | 99.0 | 84.2 |
| 8 | 98.8 | 93.5 |
| 10 | 99.9 | 98.3 |
| 12 | 100.0 | 100.0 |

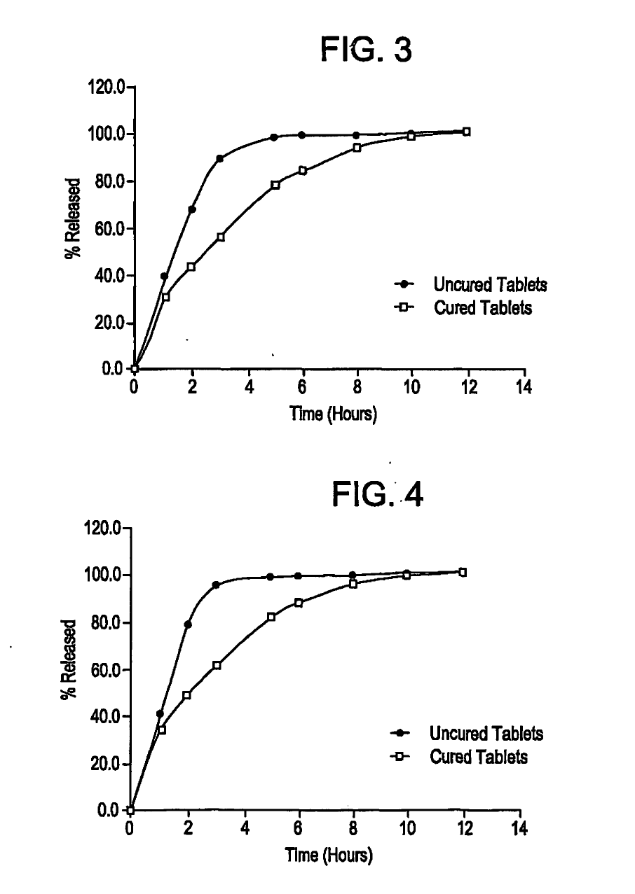

The dissolution data shown in Table 3A and illustrated in FIG. 3 showed that slower release profiles were obtained with cured tablets as opposed to uncured ones.

### Example 4

TABLE 4
Formulation 4

| | Tablet |
|---|---|
| Description | Composition (mg) |
| Oxycodone Hydrochloride | 20.000 |
| Microcrystalline Cellulose | 53.440 |
| Ammonio Methacrylate Copolymer | 68.850 |
| Colloidal Silicon Dioxide | 3.400 |
| Sodium Lauryl Sulfate | 6.800 |
| Magnesium Hydroxide | 0.510 |
| Povidone | 12.750 |
| Stearic Acid | 2.125 |
| Magnesium Stearate | 2.125 |
| Total Tablet Weight | 170.000 |
| Opadry Cosmetic Coating | 5.100 |
| Total Coated Tablet Weight | 175.100 |

TABLE 4A
Dissolution Profiles of Uncured and Cured Formulation 4 Tablets:

| | Uncured Tablets | Cured Tablets |
|---|---|---|
| | % Active Ingredient | % Active Ingredient |
| Time (hr) | Released | Released |
| 0 | 0.0 | 0.0 |
| 1 | 41.1 | 34.4 |
| 2 | 78.9 | 48.6 |
| 3 | 95.3 | 61.1 |
| 5 | 99.1 | 81.7 |
| 6 | 99.2 | 87.8 |
| 8 | 99.3 | 95.6 |
| 10 | 99.6 | 98.9 |
| 12 | 100.0 | 100.0 |

The dissolution data shown in Table 4A and illustrated in FIG. 4 showed that slower release profiles were obtained with cured tablets as opposed to uncured ones.

### Example 5

TABLE 5
Formulation 5

| | Tablet |
|---|---|
| Description | Composition (mg) |
| Oxycodone Hydrochloride | 80.000 |
| Microcrystalline Cellulose | 49.305 |
| Ammonio Methacrylate Copolymer | 132.500 |
| Colloidal Silicon Dioxide | 5.300 |
| Sodium Lauryl Sulfate | 10.600 |
| Magnesium Hydroxide | 0.794 |
| Povidone | 19.875 |
| Stearic Acid | 3.313 |
| Magnesium Stearate | 3.313 |
| Total Tablet Weight | 305.000 |
| Opadry Cosmetic Coating | 9.150 |
| Total Coated Tablet Weight | 314.150 |

TABLE 5A
Dissolution Profiles of Uncured and Cured Formulation 5 Tablets:

| | Uncured Tablets | Cured Tablets |
|---|---|---|
| | % Active Ingredient | % Active Ingredient |
| Time (hr) | Released | Released |
| 0 | 0.0 | 0.0 |
| 1 | 43.7 | 37.4 |
| 2 | 65.8 | 54.4 |
| 3 | 80.3 | 68.2 |
| 5 | 97.4 | 89.0 |
| 6 | 98.9 | 94.9 |
| 8 | 99.8 | 99.3 |
| 10 | 99.9 | 100.2 |
| 12 | 100.0 | 100.0 |

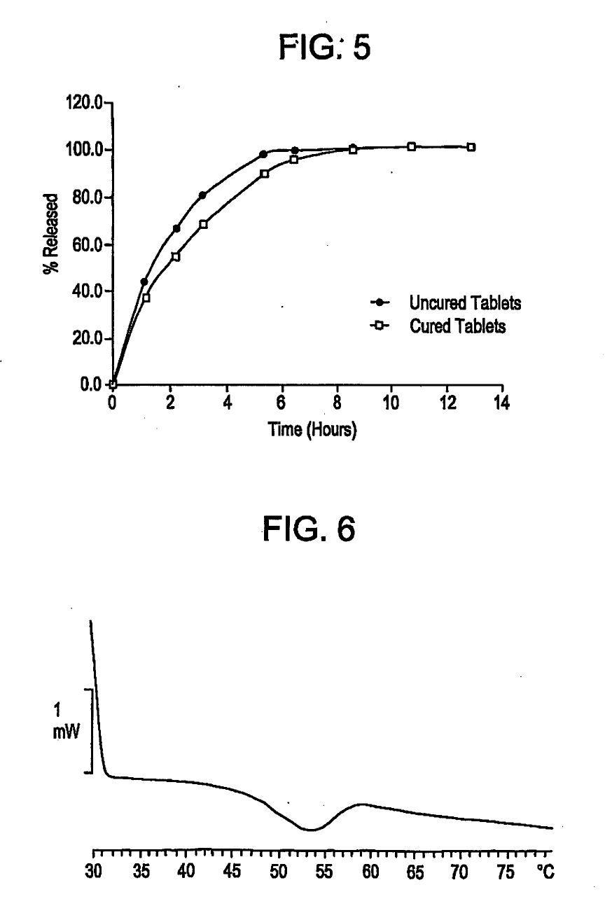

The dissolution data shown in Table 5A and illustrated in FIG. 5 showed that slower release profiles were obtained with cured tablets as opposed to uncured ones.

### Example 6

Differential Scanning Calorimetry (DSC) was used to detect physical changes of a polymer as a function of temperature. The DSC scan of the pure polymer, has a broad peak around 50° C. (FIG. 6). DSC scans of uncured tablets of formulation 1 and 2 showed similar peaks in the same region (FIGS. 7 & 9).

1. A process of preparing a controlled release oral dosage form comprising:
(a) mixing an active pharmaceutical ingredient and an acrylic polymer to yield a mixture;
(b) forming said mixture into a solid unit dosage form, and
(c) curing said solid unit dosage form.

2. The process of claim 1, wherein the active pharmaceutical ingredient is selected from the group consisting of morphine, hydromorphone, codeine, oxymorphone, nalbuphine, hydrocodone, dihydrocodeine, dihydromorphine, buprenorphine, oxycodone, naltrexone, naloxone, and pharmaceutically acceptable salts thereof.

3. The process of claim 1, wherein the acrylic polymer is ammonio methacrylate copolymer.

4. The process of claim 1, wherein the acrylic polymer comprises of about 10% to about 90% of the weight of said mixture.

5. The process of claim 4, wherein the acrylic polymer comprises of about 30% to about 70% of the dry weight of said mixture.

6. The process of claim 1 wherein the step of forming said mixture into a solid unit dosage form comprises dry granulating said active pharmaceutical ingredient with said acrylic polymer.

7. The process of claim 1 wherein the step of forming said solid unit dosage form comprises compressing said mixture.

8. The process of claim 1 wherein said solid unit dosage form is a tablet.

9. A process of preparing a controlled release oral dosage form comprising:
(a) mixing oxycodone and ammonio methacrylate copolymer to yield a mixture;
(b) forming said mixture into a tablet using dry granulation or direct compression; and
(c) curing said tablet for a time and at a temperature sufficient such that a DSC scan will produce no significant peaks in the region of from about 40° C. to about 70° C.

10. A controlled release oral dosage form produced according to the process comprising:
(a) dry mixing ant active pharmaceutical ingredient and an acrylic polymer to yield a mixture;
(b) forming said mixture into a solid unit dosage form; and
(c) curing said solid unit dosage form.

11. A controlled release oral dosage form produced according to the process comprising:
(a) dry mixing oxycodone hydrochloride and ammonio metacrylate copolymer to yield a mixture;
(b) forming said mixture into a tablet using dry granulation or direct compression; and
(c) curing said tablet at a temperature between about 40° C. and about 70° C.

12. A controlled release oral dosage form comprising an active ingredient dispersed in a sustained release matrix comprising an acrylic polymer, wherein said dosage form has been cured.

13. The controlled release oral dosage form of claim 12, comprising an acrylic polymer that exhibits no significant peaks in the region of from about 40° C. to about 70° C. on a DSC scan.

14. The controlled release oral dosage form of claim 12, wherein said acrylic polymer exhibits no significant peaks in the region of from about 46° C. to about 64° C. on a DSC scan.

15. A controlled release oral dosage form comprising an active pharmaceutical ingredient and a substantially uniform matrix which comprises from about 30% to about 70% of a cured ammonio methacrylate copolymer.
